(19) United States
(12) Patent Application Publication
(10) Pub. No.: US 2021/0228322 A1
(43) Pub. Date: Jul. 29, 2021

(54) ABUTMENT AND HEALING CUFF DEVICE FOR DENTAL IMPLANT

(71) Applicant: Gary FINELLE, Paris (FR)

(72) Inventor: Gary FINELLE, Paris (FR)

(21) Appl. No.: 15/733,941

(22) PCT Filed: Jul. 2, 2019
(86) PCT No.: PCT/FR2019/051636

§ 371 Date: Dec. 1, 2020

(30) Foreign Application Priority Data
Jul. 3, 2018 (FR) 1856141

(51) Int. Cl. A61C 8/00; A61C 8/02

(52) U.S. Cl. CPC A61C 8/0068; A61C 2201/00; A61C 8/0006; A61C 8/0089

(57) ABSTRACT

The invention relates to an intermediate dental abutment (2), intended to be inserted into a dental implant (1), the intermediate abutment (2) comprising: a lower part (20), intended to be inserted into an implant (1); an intermediate part (28), comprising an external connection device (29) for connecting a blanking element (4) of anatomical shape to close off the site of the post-extraction cavity; an upper part (24) comprising a fixing device (304) for fixing a member (9, 100), such as an impression abutment (9) or a prosthetic abutment (100).

The invention relates to the field of dental implantology.

Dental replacement techniques are known today which use a biocompatible implant device. This biological concept is based on the process of osseointegration of an implant into the bone structure, on which a dental prosthesis is fixed.

Known clinical protocols comprise several steps spaced out over time.

The first step is to extract the natural tooth to be replaced. After this extraction, a period of at least several weeks is allowed to elapse for the gum and bone to heal.

The second step, after bone healing and gingival closure, consists of placing an implant in the patient's bone structure, which successively involves making an incision in the gum, elevating the flap and drilling the bone so as to prepare the implant bed. After the implant is placed, a protective device, which can be buried (cover screw) or not buried (healing cap or healing abutment), is inserted on the head of the implant. Such a protective device makes it possible to keep the assembly isolated from chewing forces during the bone healing process, which is called osseointegration. The site is closed using a conventional suture system allowing the gingival margins to be brought together, which fit around the healing abutment. The whole remains untouched until the implant has fully healed in the bone structure by osseointegration and the gum has fully healed around the healing abutment, i.e. for a period of several months.

The third step consists in taking an impression, that is to say, in recording the position of the implant and of the surrounding anatomical structures (adjacent teeth, gums, antagonistic arch, etc.), in order to produce an anatomical implant crown. The impression is taken by removing the healing abutment and inserting an impression abutment (also called impression coping) into the implant; this impression abutment can be of the digital type (reference is then generally made to scan bodies) or of the conventional type, depending on the impression taking method used by the practitioner (impression taking by three-dimensional digital scanning, or manual or conventional impression taking). After the impression is taken, the healing abutment is screwed back to the implant during the manufacturing time of the prosthesis, pending the installation step of the latter.

The fourth and final step is the placement of the dental prosthesis, which is carried out by screwing an implant-prosthetic structure in one part (in the case of a screw-retained rehabilitation) or in two parts (in the case of a sealed rehabilitation).

Conventional protocols, such as the one described above, have the drawback of increasing the number of procedures on the patient, and in particular of requiring several procedures on the gum after it has healed following the placement of the implant. In addition, the extraction of the natural tooth and the placement of the implant are generally carried out during two independent procedures and spaced apart by several months, in order to allow the bone site to regenerate, both at the bone and mucous levels. These various procedures involve surgical management of an incompressible and relatively long duration, spanning several months. In contrast, each procedure presents an operative risk linked to the surgical procedure itself. Thus, the multiplicity of procedures increases the invasiveness of the treatment for the patient as well as the risks of postoperative treatment.

In order to limit the number of procedures, techniques have been developed which consist in performing the steps of extracting the tooth and placing the implant during the same procedure. However, the morphology of each extraction socket being specific, and the dimensions of the surgical site being larger than the industrialized healing caps, obtaining the closure of the operating site forces clinicians to perform invasive surgical manipulations, as well as significant flap displacements, which generates tissue resorption phenomena and often painful post-operative effects.

There is therefore a need to implement protocols that can simplify care, reduce the invasiveness of the treatment and limit procedures on the patient from the extraction of the tooth to the fixation of the final implant prosthesis.

Thus, the aim of the present invention is to remedy the drawbacks of the state of the art, and more particularly those set out above, by proposing a peri-implant healing cuff which makes it possible to:
improve gingival healing by preventing gum collapse due to the lack of mechanical support (after extraction);
stabilize the blood clot in an enclosed space favorable to bone regeneration;
limit the number of procedures on the patient;
limit the duration of treatment;
limit manipulations on the implant and/or the elements secured to the implant.

To this end, the invention relates to an intermediate dental abutment, intended to be inserted into a dental implant, the intermediate abutment comprising:
a lower part, intended to be inserted into an implant;
an intermediate part, comprising an external connection device for connecting a closure element of anatomical shape to close off the site of the post-extraction cavity;
an upper part, comprising a fixing device for fixing a member, such as an impression abutment or a prosthetic abutment.

Thus, by providing an intermediate abutment that allows the fixing of anatomical closure elements of various shapes and sizes, which can be customized if necessary, the invention makes it possible to ensure the healing of the post-extraction surgical site by faithfully reproducing the morphology of the emergence of the natural tooth which was initially present. The intermediate abutment in accordance with the invention in fact makes it possible to connect, at the intermediate part of the abutment (which corresponds to the transmucosal part of the abutment, that is to say, the part of the abutment which is located in the gingival area of the surgical site when the abutment is positioned on an implant inserted into the bone structure), a biocompatible anatomical closure element so as to ensure the closure of the site of the post-extraction cavity (i.e. the socket formed by the gingival contour immediately after extraction of the original tooth). This element can thus be chosen from a set covering the different cervical morphologies of the different teeth of the dental arch, thus making the healing cuff (formed by the assembly of the intermediate abutment and of a closure element in accordance with the invention) fully adaptable during the procedure and customizable to a perfectly anatomical shape, which shape is determined by the gingival contour immediately after extraction.

If necessary, a final adaptation of the closure element will be done, in vivo, at the surgical site using a composite restorative resin (e.g. a light-curing resin). If necessary, also, the closure element can be cut again for a very fine adaptation to the surgical site (for example by being cut with scissors or milled with a burr by the dental surgeon).

The intermediate abutment according to the invention also has a connection device on its occlusal (or upper) part allowing an element such as an impression abutment or a prosthetic element (temporary tooth, for example) to be fixed above the anatomical closure element. Thus, it is not necessary to remove either the anatomical closure element or the intermediate abutment in order to take the impression for the manufacture of a prosthesis. This limits the number of elements that must be successively connected to the implant. Finally, the abutment according to the invention can also serve as an intermediate abutment supporting the final prosthetic abutment. In such a configuration, the abutment according to the invention is never removed after its initial placement, which limits procedures on the patient and thus reduces the risks and trauma associated with the procedures, in particular with regard to the lesion from the biological attachment between the peri-implant mucosa and the prosthetic abutment.

The external arrangement of the connection device of the closure element makes it possible to make this connection device accessible from the lateral (outer) surface of the abutment, for external (or lateral) fixation of the closure element to the abutment. External or lateral fixation in particular means that the fixing device is accessible even when the intermediate abutment is connected to an implant. Thus, the closure element can be fixed to the intermediate abutment (and, conversely, removed from the latter) while the intermediate abutment is already fixed to an implant which has previously been inserted into the bone structure of the patient. The intermediate abutment is therefore configured so that the external connection device for connecting an anatomically-shaped closure element remains accessible when the abutment is fixed to an implant. Further, the intermediate abutment is configured so that the fixing device which is present on its upper part remains accessible even when a closure element is attached to the intermediate abutment.

Advantageously, the closure element is fixed to the external connection device of the intermediate abutment by shape cooperation, and in particular by elastic interlocking or clipping. The closure element can thus be easily linked to the intermediate abutment in vivo, in a reliable manner, without requiring fasteners such as a screw.

In one embodiment, the external connection device is configured to allow the fixing of a closure element by shape cooperation, in particular by clipping.

In one embodiment, the external connection device for connecting a closure element comprises at least one generally circular groove formed on the external surface of the intermediate part of the intermediate abutment.

In one embodiment, the intermediate abutment comprises at least two superimposed grooves.

In one embodiment, the intermediate abutment comprises at least three superimposed grooves.

In one embodiment, the groove(s) is (are) continuous around the entire periphery of the intermediate part of the intermediate abutment.

In one embodiment, the groove(s) is (are) discontinuous around the periphery of the intermediate part of the intermediate abutment, and comprise a plurality of adjacent sectors, for example two, four, six or eight adjacent sectors.

In one embodiment, the intermediate abutment is configured so that the intermediate part is, when the latter is connected to an implant placed in the bone structure of a patient, located in the gingival area of the surgical site, that is to say, in the transmucosal part.

In one embodiment, the device for fixing a member comprises an internal thread.

In one embodiment, the device for fixing a member is associated with a device for indexing the position of the member relative to the intermediate abutment, and in particular the angular position of the member.

In one embodiment, the intermediate abutment comprises a screw allowing the intermediate abutment to be fixed in an implant, in particular a captive screw.

In one embodiment, the intermediate abutment comprises a through bore, a first portion of the bore being configured to allow the passage of the screw, a second portion of the bore comprising the device for fixing a member.

The invention also relates to a healing cuff comprising an intermediate abutment as defined above and an anatomical closure element, the closure element comprising a fixing part intended to cooperate with the external connection device of the intermediate abutment, and a closure part having an anatomical shape.

In one embodiment, the fixing part of the closure element comprises one or more resiliently deformable tongues which are configured to cooperate with the external connection device of the intermediate abutment.

In one embodiment, the closure element comprises a biocompatible material, in particular a biocompatible polymer, such as polyetheretherketone (or PEEK) or polyurethane.

In one embodiment, the closure part has an outer edge of serrated shape and/or comprising a surface exhibiting a roughness promoting the adhesion of a resin, in particular a light-curing composite resin.

The invention also relates to an assembly of an intermediate abutment as defined above and of a prosthetic abutment for an implant prosthesis, the prosthetic abutment being connected to the intermediate abutment, the prosthetic abutment comprising for this purpose a tubular section shaped to at least partially surround the intermediate part of the intermediate abutment when the prosthetic abutment and the intermediate abutment are connected via the fixing device of the intermediate abutment.

The invention also relates to a supragingival implant planning kit comprising an intermediate abutment as defined above and several closure elements as defined above.

The invention also relates to a tool for selecting a closure element as defined above, characterized in that the tool comprises one or more templates reproducing the two-dimensional profile of a closure element (or of the assembly formed by an intermediate abutment and a closure element), or the three-dimensional shape of a closure element (or the set of an intermediate abutment and a closure element).

The present invention will be better understood on reading the following detailed description, given with reference to the accompanying drawings, in which:

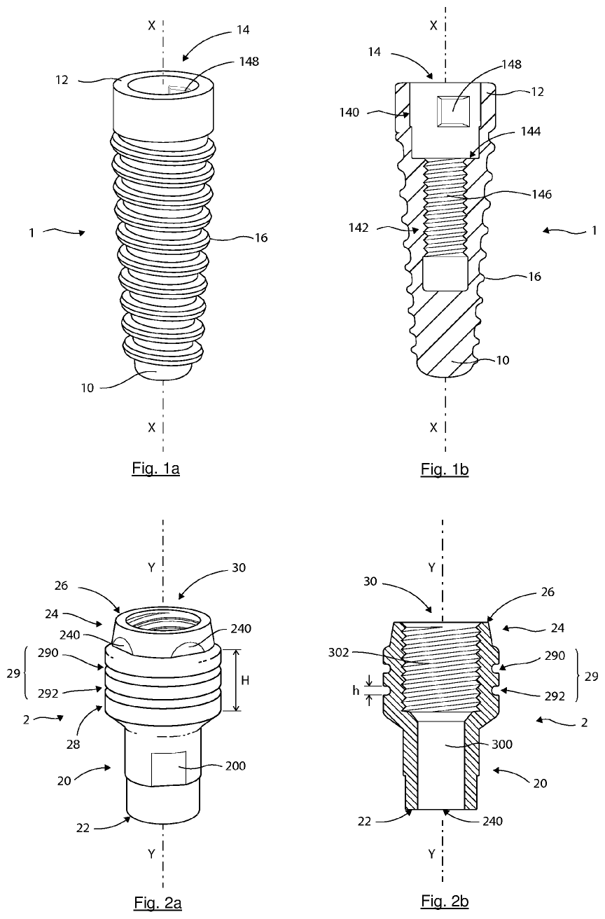

FIGS. 1a and 1b show an implant, seen in perspective and in section, respectively;

FIGS. 2a and 2b show an intermediate abutment according to the invention, seen respectively in perspective and in section;

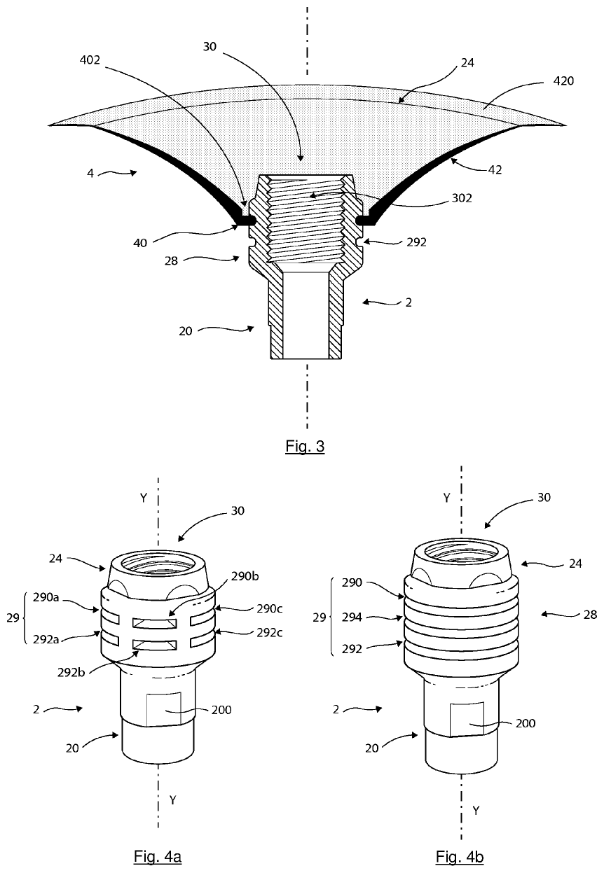

FIG. 3 is a sectional view of the intermediate abutment of FIGS. 2a and 2b equipped with a closure element according to the invention;

FIGS. 4a and 4b are perspective views of a second and a third embodiment of an intermediate abutment according to the invention;

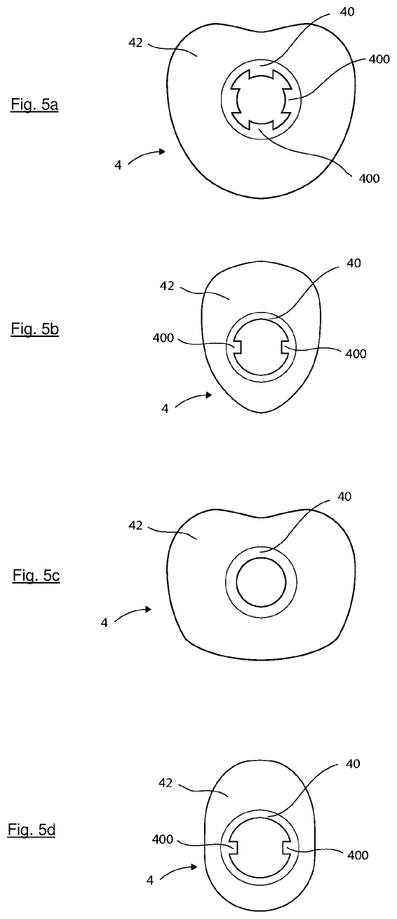

FIGS. 5a to 7f illustrate different shapes of a closure element according to the invention, according to the different dental anatomies;

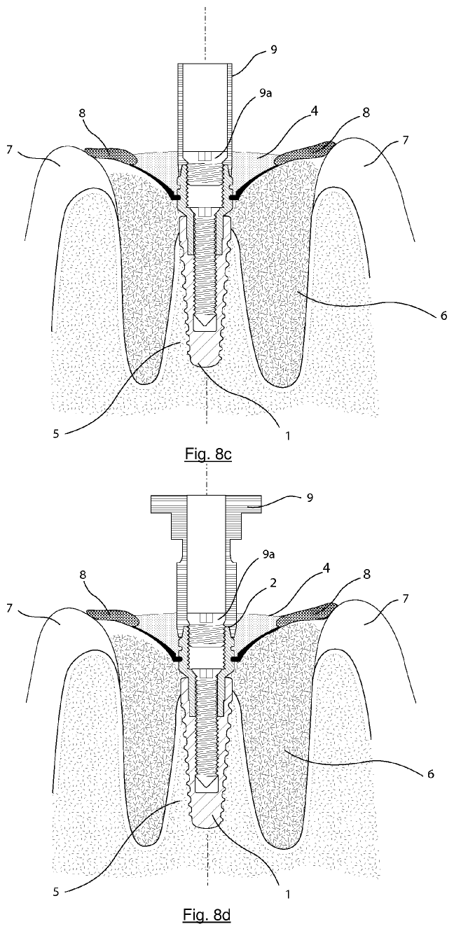

FIGS. 8a to 8d illustrate various stages of the fitting of a dental prosthesis using an intermediate abutment according to the invention;

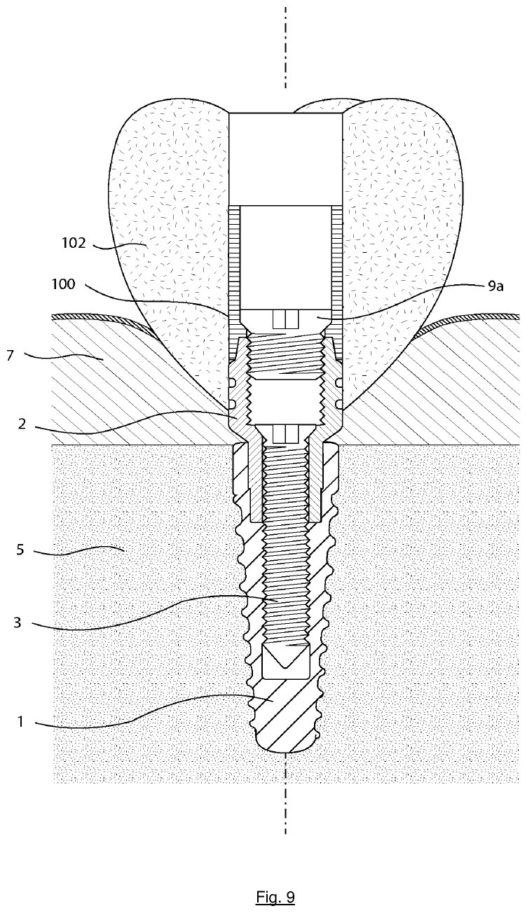

FIG. 9 is a sectional view of the gum of a patient, in which a dental prosthesis is fixed to an intermediate abutment according to the invention;

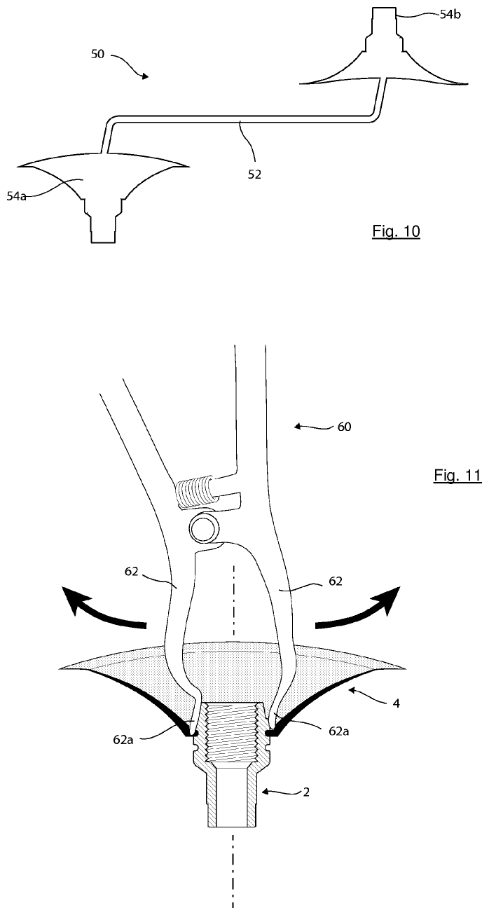

FIGS. 10 and 11 illustrate two tools to facilitate the use of an intermediate abutment according to the invention.

FIGS. 1a and 1b show an example of a dental implant, respectively seen in perspective and in section. The implant 1 shown in FIGS. 1a and 1b is, in a known manner, configured to be fitted and mechanically stabilized by a screwing technique into the jawbone or mandibular bone of a patient. After a healing time of several weeks, the mechanical stability is replaced by a more reliable biological stability, the implant is then osseointegrated, and the placement of the final prosthesis can then be considered. In the example, the implant 1 is of generally frustoconical shape with axis X (but can of course be of any other suitable shape, for example cylindrical). The implant 1 has a first end 10, or apical end, which is closed, and a second end 12, or cervical end, opposite the first end, and at which a blind bore 14 opens that is made in the body of the implant 1. The implant 1 has an external thread 16, which may or may not be a self-tapping-type thread. The bore 14 comprises a first portion 140, which is not threaded. The first portion is connected, via a shoulder 144, to a second portion 142, which has an internal thread 146. The first portion 140 of the bore 14 comprises at least one internal indexing element 148, which is intended to cooperate with a corresponding shape provided on an element to be connected to the implant 1, such as an intermediate abutment. In the example, the indexing element 148 forms a protuberance from the surface of the first portion 140 of the bore 14.

FIGS. 2a and 2b show an intermediate abutment 2 according to the invention, seen respectively in perspective and in section. The intermediate abutment 2 is made of any suitable biocompatible material, such as titanium, polyetheretherketone (or PEEK), or a ceramic material such as zirconia, for example. The intermediate abutment 2 has a general shape which is tubular and generally symmetrical in revolution with axis Y. The intermediate abutment 2 has a first part 20 (or lower part 20), a free end of which forms the lower end 22 of the intermediate abutment, the first part 20 being shaped to be inserted into a dental implant such as the implant 1 of FIGS. 1a and 1b. The opposite end of the intermediate abutment, or upper end 26, is formed by the free end of a second part 24, or upper part 24, of the intermediate abutment 2.

An intermediate part 28 is arranged between the lower 20 and upper 24 parts. The intermediate abutment 2 is configured so that the intermediate part 28 corresponds to the transmucosal part of the abutment, that is to say that when the intermediate abutment 2 is positioned on an implant placed in the bone structure of a patient, then the intermediate portion 28 is located in the gum area of the surgical site.

The intermediate abutment 2 has a through bore 30, opening out at the upper 26 and lower 22 ends of the intermediate abutment. The bore 30 constitutes a screw access hole.

The bore 30 has a first portion 300, opening out into an orifice 220 located at the lower end 22 of the intermediate abutment 2. The first portion 300 is intended to allow the passage of a fixing screw ensuring the fixing of the intermediate abutment to the implant 1.

The first portion 300 of the bore 30 is topped by a second portion 302, the internal diameter of which is greater than the internal diameter of the first portion 300. The second portion 302 comprises an internal thread 304 allowing the fixing of an external member, in particular by means of a screw.

On the outer surface of the lower part 20, the intermediate abutment 2 comprises an indexing element 200, the shape of which is complementary to that of the indexing element 148 of the implant 1, thus allowing an indexed angular positioning of the intermediate abutment 2 in implant 1.

In the example, the intermediate part 28 of the intermediate abutment 2 is of generally cylindrical shape with axis Y. According to the invention, the intermediate part 28 comprises at least one external connection device 29 for connecting an anatomical closure element. In the example, the intermediate part comprises an external connection device 29 with two levels (or stages), in the form of two superimposed circular grooves 290, 292, i.e. an upper groove 290 and a lower groove 292.

As mentioned above, the intermediate part 28 is arranged between the lower 20 and upper 24 parts of the intermediate abutment 2. Thus the intermediate part 28 is topped by the upper part 24, which in the example is generally frustoconical with axis Y. On the outer surface of the upper part 24, the intermediate abutment 2 comprises a plurality of external indexing elements 240, which in the example are flats 240, allowing the indexing of an element fixed to the intermediate abutment 2. In the example, four flats are provided which are evenly distributed around the periphery of the upper part 24, but alternatively a different number of indexing elements can be provided, for example six or eight. One or more internal indexing elements, arranged within the bore 30, could also be provided, alternatively or in addition.

As shown in FIG. 3, owing to the grooves 290, 292 formed in the outer surface of the intermediate part 28 of the intermediate abutment 2, an anatomical closure element 4 can be simply and easily fixed, in a reliable manner, to the intermediate abutment 2 by shape cooperation, for example by clipping. For this purpose, the closure element 4 comprises a fixing part 40 of generally annular shape, the fixing part 40 being able to cooperate by shape cooperation, in particular by clipping, with one of the grooves 290, 292 of the intermediate abutment 2. In the example, the fixing part 40 comprises one or more tongues 400 (visible in particular in FIGS. 5a to 5d) for this purpose which are radially oriented, resiliently deformable and able to be inserted into one of the grooves 290, 292 of the intermediate abutment 2 so as to ensure the retention of the closure element 4 by resilient fitting and/or clipping. Advantageously, the fixing part 40 comprises a tubular part defining an annular space 402 between the fixing part 40 and the intermediate abutment 2. Thus, this annular space 402 can, if necessary, be filled with resin in order to secure the fixing of the closure element 4 on the intermediate abutment 2. The fixing part 40 is connected to an anatomical closure part 42, the shape and dimensions of which can be finely adapted to the anatomy of the patient who is to receive the healing cuff formed by the intermediate abutment 2 and the closure element 4. The closure part 42 is advantageously in the general shape of a collar, and in particular has a flared shape from the fixing part 40 to an outer edge 420. In the example, the outer edge 420 of the closure part 42 has a substantially flat shape. Advantageously, the outer edge 420 will have a surface with a high roughness (for example a sandblasted surface), so as to promote the adhesion of the resin. Alternatively or in addition, provision may be made for the outer edge 420 to have a serrated or crenellated shape, in order to allow improved adhesion of the biocompatible resin which will be deposited at this outer edge, as will be seen below. The closure element 4 will be made from any suitable biocompatible material, such as, for example, PEEK or a biocompatible polymer such as polyurethane. The thickness of the fixing part 40 will in particular be between 0.4 and 1.5 millimeters, and for example equal to 0.5 millimeters. The thickness of the closure part 42 may for example be between 0.5 and 4 millimeters. The outer edge 420 of the closure part will for example be between 0.5 and 2.5 millimeters.

By way of example, the external diameter of the intermediate part 28 of the abutment 2 may be between 3 and 6 millimeters, and will depend in particular on the diameter of the implant. The height H of the intermediate part may be between 1 and 5 millimeters. The height of the grooves 290, 292 may for example be between 0.4 and 1.5 millimeters, and for example equal to 0.5 millimeters. In the case of an abutment comprising several fixing stages, the grooves will for example be spaced apart by a value h of between 0.5 and 1.5 millimeters, and for example equal to 1 millimeter.

The main steps of the use of an intermediate abutment 2 and of a closure element 4 according to the invention are described below, in particular in relation to FIGS. 8a to 8d.

FIG. 8a shows the gum of a patient seen in section, in which, successively:
the bone structure 5 has been inserted into the implant 1;
a bone substitute material 6 has been used to fill the post-extraction socket, in order to preserve bone volume and prevent resorption during healing periods;
an intermediate abutment 2 according to the invention of suitable size has been selected, then inserted and secured to the implant 1 by means of a fixing screw 3. The intermediate abutment will in particular be chosen so that once it is fixed to the implant, the intermediate part 28 is located in the transmucosal part of the patient;
a closure element 4 has been selected and positioned on the intermediate abutment 2 whose shape, size and positioning height (in the case where the intermediate abutment 2 has an external connection device at several levels) are chosen so as to correspond as closely as possible to the anatomy of the patient's alveolar emergence. In particular, it is observed that the closure element 4 has a shape and a height (that is to say the dimension along the axis of revolution Y of the intermediate abutment) allowing the outer edge of the closure part 42 to come close to the opening of the marginal gingiva 7 of the patient. Thus, after the procedure of extraction, implantation, and placement of the intermediate abutment, the closure element 4 makes it possible to partially close the alveolar site, initially leaving only a small space between the abutment and the marginal gingiva 7 (e.g. a space of one to two millimeters). Of course, the closure element 4 may, if necessary, be re-cut by the practitioner so as to make it slightly undersized with respect to the dimensions of the socket.

FIG. 8b shows the final step of closing the gum tissue after positioning the intermediate abutment 2 and the closure element 4. This step consists in closing the alveolar site, by adding a biocompatible material 8 of the composite resin type (in particular a light-curing resin), making it possible to create a hermetic barrier between the surgical site and the oral cavity, thus reducing the risk of contamination and passage of undesirable exogenous elements (food, for example). The healing cuff according to the invention plays an important role in the mechanical support of the post-extraction gingival architecture and in the transmucosal gingival arrangement. It also plays a role in the stability of the blood clot and in the biological response associated with the process of bone regeneration.

It should be noted that a practitioner equipped with a computer-aided manufacturing system can advantageously produce a suitable closure element on site, after taking an impression of the patient's surgical site. Manufacturing can then be carried out by additive or subtractive manufacturing techniques.

As can be seen in FIGS. 8a to 8d, it will be noted that the external positioning of the closure element 4 allows simple fixing, without additional elements (such as a screw).

It is therefore understood that the invention makes it possible to greatly simplify the implementation of the immediate extraction/implantation protocols, by proposing a solution for closing the alveolar site which is perfectly suited to each patient. Indeed, the intermediate abutment 2 allows perfect individualization of the healing cuff, adapted to each clinical situation once the tooth has been extracted. Thus, the extraction of the tooth, the steps of drilling, fitting the implant, fitting the intermediate abutment 2, the closure element 4, and, where appropriate, a layer of resin are carried out in a single procedure.

Advantageously, the intermediate abutment according to the invention is configured so as not to emerge or to emerge little beyond the cervical zone, in order to avoid the mechanical stresses that are inherent in the functions of the manducatory apparatus (chewing, swallowing, etc.). Once it is firmly fixed, the healing cuff will be left untouched, so as to allow normal healing until osteointegration is achieved. This step usually takes a few months.

Once the healing step has been completed, it is necessary to take an impression of the three-dimensional position (including indexing) of the implant in relation to the anatomical and dental structures of the patient (which includes the position of the gum as stabilized around the healing cuff). This impression will make it possible to generate a working model from which the prosthesis will be made. According to the invention, the intermediate abutment 2 is configured so that the impression-taking step can be carried out without disassembling the intermediate abutment 2 (and without removing the anatomical closure element 4), unlike the known methods using conventional healing caps (these healing caps must be removed to make room for an impression abutment). As can be seen in FIGS. 8c and 8d, which respectively illustrate a digital impression and a conventional impression, neither the disassembly of the intermediate abutment 2 nor that of the closure element 4 is required to connect an impression abutment, since the upper part 24 of the intermediate abutment, including the fixing device 304, remains accessible even in the presence of an anatomical closure element 4. The fixing of an impression abutment 9, of the digital type (FIG. 8c) or of the conventional type (FIG. 8d), is carried out by means of a fixing screw 9a, which cooperates with the internal thread 304 of the bore 30 of the intermediate abutment 2. In addition, the indexing elements 240 cooperate with indexing elements of complementary shape present on the impression abutment 9, and which allow an indexed angular positioning of the latter.

Once the impression has been taken, one step consists in manufacturing the dental prosthesis using the data collected during the impression taking step.

The final step is to install the dental prosthesis, visible in FIG. 9. The dental prosthesis can be manufactured in order to be screwed directly to the implant platform; in this case the healing assembly formed by the intermediate abutment 2 and the closure element 4 is deposited on the day of the final prosthetic assembly. In the example of FIG. 9, it has been envisaged to keep the intermediate abutment 2, which then constitutes an intermediate abutment for fixing a prosthetic abutment 100. The dental prosthesis 102 is fixed using the prosthetic abutment 100, the latter being fixed in the intermediate abutment 2, by means of a fixing screw 9a which cooperates with the internal thread 304, after removing the closure element 4. This configuration has the particular advantage of never requiring the removal of the intermediate abutment 2, from its initial insertion into the implant 1. This makes it possible to further reduce the manipulations required during the dental restoration. In the example, the dental prosthesis 102 is shaped so as to at least partially surround the intermediate part 28 of the intermediate abutment 2, and covers the external connection device 29. Advantageously, the prosthetic abutment 100 may comprise a tubular section (not shown) extending so as to surround the intermediate part 28 at least partially, preferably entirely. In such a configuration, the tubular section surrounds at least the external connection device 29.

FIGS. 5a to 7f show different shapes of the closure element 4.

As can be seen in FIGS. 5a, 5b, 5c and 5d, the shape of the closure element 4 is advantageously adapted to the type of the tooth which must be replaced. At least four different shapes of the closure element 4 will therefore advantageously be provided: a first shape corresponding to a maxillary molar (FIG. 5a), a second shape corresponding to a mandibular molar (FIG. 5b), a third shape corresponding to a premolar (FIG. 5c) and a fourth shape corresponding to an incisor (FIG. 5d).

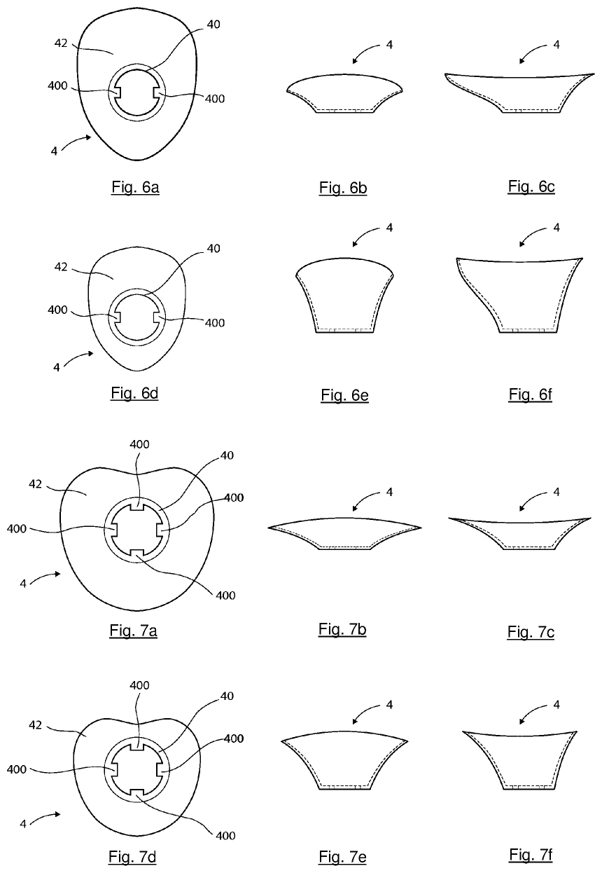

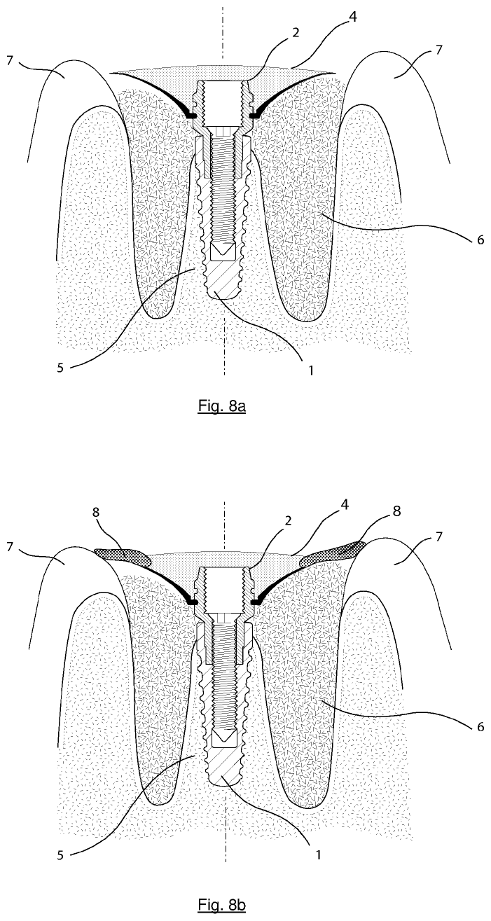

Advantageously, for each of the different shapes, several different sizes can be provided. For example, FIGS. 6a and 6d show two examples of different sizes of the first shape (corresponding to a maxillary molar), while FIGS. 7a and 7d show two examples of different sizes for the fourth shape (corresponding to an incisor).

Also advantageously, for each size and each shape, different heights and profiles will be provided, as shown in FIGS. 6b, 6c, 6e and 6f for the first shape, and in FIGS. 7b, 7c, 7e and 7f for the fourth shape.

FIGS. 3a and 3b show two embodiments of the intermediate abutment according to the invention.

In the example of FIG. 3a, the grooves 290, 292 are discontinuous, thus being formed by a plurality of adjacent sectors 290a, 290b, 290c and 292a, 292b, 292c. In the example, each groove is formed by four sectors, but alternatively, a lower number (for example two) or a higher number (for example six or eight) of sectors can be provided. The discontinuous grooves of FIG. 3a make it possible in particular to envisage indexing the angular position of the closure element, on which one or more tongues 400 of complementary shape will be provided.

In the example of FIG. 3b, the intermediate abutment 2 has three grooves 290, 292 and 294. Providing three grooves instead of two makes it possible to increase the possibilities for positioning the closure element 4 in terms of height, and to better adapt to the anatomy of the patient. It will be noted that in order to simplify the abutment 2, a single groove can also be provided. Regardless of the number of grooves provided, provision may be made for these to be continuous or discontinuous.

FIGS. 10 and 11 show examples of tools that can help the practitioner during procedures implementing an intermediate abutment according to the invention.

FIG. 10 illustrates an example of a tool 50 which is capable of helping the practitioner in the selection of a closure element 4 that is suitable for the patient. In the example, the tool comprises a gripping rod 52 secured to one or more shapes 54a, 54b that are representative of the profile of the assembly formed by a closure element associated with an intermediate abutment according to the invention. These shapes constitute templates allowing the practitioner to determine, for a given intermediate abutment, which closure element (depending on its size and shape) to associate with this abutment, and, when the intermediate abutment comprises an external connection device 29 with several levels (like in FIGS. 3, 4a and 4b), to which level of the pillar to fix the closure element. In the example, the tool 50 is shown with two shapes 54a, 54b, representing closure elements with different heights. Thus, owing to the tool 50, the practitioner can test in situ which closure element will best correspond to the post-extraction site, without having to open several (real) closure elements, these being packaged in sterile packaging. Advantageously, the tool 50 could also make it possible to select the shape and/or the size of the intermediate abutment to be used. In the example of FIG. 10, the tool comprises shapes 54a, 54a which are flat and constitute two-dimensional templates. It would of course be possible to provide shapes constituting three-dimensional templates.

FIG. 11 shows an example of a tool 60 for gripping and inserting a closure element 4 positioned on an intermediate abutment 2. In the example, the tool 60, which is in the form of a spacer clamp, comprises two arms 60 that are articulated to one another, the free end 62a of each arm being configured to be inserted between the closure element 4 and the intermediate abutment 2. Thus, the action of the tool 60 will make it possible to separate the fixing part 40 from the closure element, so as to release the tongues 400 from the grooves of the intermediate abutment 2. Advantageously, the tool 60 may have more than two arms, for example three or four arms, these arms being configured to move apart in angularly evenly distributed directions, in order to improve the distribution of the separation forces exerted on the fixing part of the closure element.

Advantageously, the intermediate abutment according to the invention will be supplied with a kit comprising a certain number of healing elements, of different shapes, sizes and heights.

1. Intermediate dental abutment (2), intended to be inserted into a dental implant (1), the intermediate abutment (2) comprising:
a lower part (20), intended to be inserted into an implant (1);
an intermediate part (28), comprising an external connection device (29) for connecting a closure element (4) of anatomical shape to close off the site of the post-extraction cavity;
an upper part (24), comprising a fixing device (304) for fixing a member (9, 100), such as an impression abutment (9) or a prosthetic abutment (100).

2. Intermediate abutment (2) according to claim 1, wherein the external connection device (29) is configured to allow the fixing of a closure element (4) by shape cooperation, in particular by clipping.

3. Intermediate abutment (2) according to claim 2, wherein the external connection device (9) for connecting a closure element (4) comprises at least one generally circular groove (290, 292, 294) formed on the external surface of the intermediate part (28) of the intermediate abutment (2).

4. Intermediate abutment (2) according to claim 3, comprising at least two superimposed grooves (290, 292).

5. Intermediate abutment (2) according to claim 4, comprising at least three superimposed grooves (290, 292, 294).

6. Intermediate abutment (2) according to claim 3, wherein the groove(s) (290, 292, 294) are continuous around the entire periphery of the intermediate part (28) of the intermediate abutment (2).

7. Intermediate abutment (2) according to claim 3, wherein the groove(s) are discontinuous around the periphery of the intermediate part (28) of the intermediate abutment (2), and comprise a plurality of adjacent sectors (290a, 290b; 290c, 292a, 292b, 292c), for example two, four, six or eight adjacent sectors.

8. Intermediate abutment (2) according to claim 1, the intermediate abutment (2) being configured so that the intermediate part (28) is, when the intermediate abutment (2) is connected to an implant (1) placed in the bone structure of a patient, located in the gingival area of the surgical site, or transmucosal part.

9. Intermediate abutment (2) according to claim 1, wherein the fixing device (304) of a member (9, 100) comprises an internal thread (304).

10. Intermediate abutment (2) according to claim 1, wherein the device (304) for fixing a member (9, 100) is associated with a device (240) for indexing the position of the member (9, 100) relative to the intermediate abutment (2), and in particular the angular position of the member (9, 100).

11. Intermediate abutment (2) according to claim 1, comprising a screw (3) allowing the intermediate abutment to be fixed in an implant (1), in particular a captive screw.

12. Intermediate abutment (2) according to claim 1, comprising a through bore (30), a first portion (300) of the bore (30) being configured to allow the passage of the screw (3), a second portion (302) of the bore (30) comprising the device (304) for fixing a member (9, 100).

13. Healing cuff comprising an intermediate abutment (2) according to claim 1 and an anatomical closure element (4), the closure element (4) comprising a fixing part (40) intended to cooperate with the external connection device (29) of the intermediate abutment (2), and a closure part (42) of anatomical shape to close off the site of the post-extraction cavity.

14. Healing cuff according to claim 13, wherein the fixing part (40) of the closure element (4) comprises one or more resiliently deformable tongues (400) which are configured to cooperate with the external connection device (29) of the intermediate abutment.

15. Healing cuff according to claim 13, characterized in that the closure element (4) comprises a biocompatible material, in particular a biocompatible polymer, such as polyetheretherketone or polyurethane.

16. Healing cuff according to claim 13, wherein the closure part (42) has an outer edge (420) of serrated shape and/or comprising a surface exhibiting a roughness promoting the adhesion of a resin, in particular a light-curing composite resin.
